(19) United States
(12) Patent Application Publication
(10) Pub. No.: US 2005/0027340 A1
(43) Pub. Date: Feb. 3, 2005

(54) System and method for providing a medical lead body having dual conductor layers

(75) Inventors: Michael P. Schrom, Wyoming Township, MN (US); Charles F. Lehman, Minneapolis, MN (US); Mark Gerald Schrom, Hugo, MN (US)

Correspondence Address: DOCKET CLERK, DM/ANSI, P.O. BOX 802432, DALLAS, TX, 75380, US

(73) Assignee: MICRONET MEDICAL, INC., White Bear Lake, MN (US)

(21) Appl. No.: 10/630,233

(22) Filed: Jul. 29, 2003

(51) Int. Cl. A61N 1/05

(52) U.S. Cl. 607/116

(57) ABSTRACT

An implantable medical lead and lead body, method of manufacturing the same, and a system and method for stimulating a portion of a body is disclosed. In one advantageous embodiment, a lead body assembly is formed by preparing a first layer unitary body comprising a first plurality of conductors. An inner layer of extrusion material is placed on the first layer unitary body. A second plurality of conductors coated with extrusion material is placed on the inner layer. An outer layer of extrusion material is placed over the second plurality of conductors. Heat shrink tubing is placed over the assembly and the assembly is heated to melt the extrusion material. The extrusion material is compressed around the conductors. The assembly is cooled and the heat shrink tubing is removed. The solidified extrusion material forms a protective wall that encapsulates the first and second plurality of conductors in the lead body.

### CROSS-REFERENCE TO RELATED PATENT DOCUMENTS

The present disclosure is related to the inventions disclosed in the following United States patent applications:

U.S. patent application No. [Attorney Docket Number 03-002] filed concurrently herewith, entitled “System and Method for Providing A Medical Lead Body”; and

U.S. patent application No. [Attorney Docket Number 03-009] filed concurrently herewith, entitled “System and Method for Providing A Medical Lead Body Having Conductors That Are Wound in Opposite Directions.”

These patent applications are commonly owned by the assignee of the present invention. The disclosures of these related United States patent applications are incorporated herein by reference for all purposes as if fully set forth herein.

### TECHNICAL FIELD OF THE INVENTION

The present invention generally relates to medical leads and, more particularly, to a system and method for manufacturing an implantable lead that includes a lead body having a first layer unitary body that comprises a first plurality of conductors and a second layer that comprises a second plurality of conductors and extrusion material.

### BACKGROUND OF THE INVENTION

Electrical signals may be used in a variety of medical applications to provide electrical stimulation to various parts of the body of a patient. For example, electrical signals may be used to modulate the amount of pain perceived by a patient by electrically stimulating a site near one or more nerves of the patient. A source of electrical signals may be implanted within the body of a patient. Electrical signals are conducted from the source of electrical signals to the stimulation site of the patient through a lead implanted within the body of the patient.

A lead generally includes a thin, flexible, lead body that contains electrically conducting conductors (e.g., wires) that extend from a first end of the lead (the proximal end) to a second end of the lead (the distal end). The lead body includes insulating material for covering and electrically insulating the electrically conducting conductors. The proximal end of the lead further includes an electrical contact that may be coupled to a source of electrical signals and the distal end of the lead includes an electrode that may be placed at the stimulation site within the body of the patient.

A prior art manufacturing process that the inventors developed for a lead involved placing a plurality of electrically conducting conductors on a layer of extrusion material placed on an underlying mandrel. This method was developed for only up to four conductors, because the conductors ran longitudinally along the length of the mandrel. Because only four wires were used, concern about insulating the conductors were minimized by evenly spacing the wires along the length, something that was simplified because of placement of the wires along the length of the mandrel. Greater than four conductors caused concern for mass production because of narrowing spacing requirements tended to cause conductor interference and shorts, since it became more difficult to evenly space the conductors.

After the conductors were in place on the extrusion material on the mandrel in this method, the conductors were then covered with another layer of extrusion material and a heat shrink process was applied to melt the extrusion material. The extrusion material was then cooled to form a lead body that encapsulated the conductors.

Different prior art conductors suggest that the conductors may be wound around a cylindrically shaped mandrel in a spiral manner. Here, a mechanical comb is utilized in the prior art winding process to keep the conductors separated as the conductors are wound around the mandrel. The use of a mechanical comb can sometimes cause the pitch of the conductors to vary. The term “pitch” refers to the distance along the axis of the mandrel that represents one turn of conductor around the mandrel.

The use of mechanical combs can also sometimes damage the conductors. Prior art manufacturing methods can also result in a lead body that has variable (non-uniform) conductor pitches for the conductors in the lead body. Prior art manufacturing methods can also result in a lead body that has variable (non-uniform) wall thicknesses. Prior art manufacturing methods also can result in the creation of lead bodies that have relatively large diameters.

Larger electrode-carrying catheters in the prior art (such as those used in cardiology applications) may utilize electrically conducting wires that are spirally wound around a cylindrically shaped wire core. For example, U.S. Pat. No. 5,417,208 issued to Winkler describes an electrode-carrying catheter that comprises insulated wires (or non-insulated wires) that are spirally wound under hand tension around a cylindrically symmetrical wire core. The wires are embedded in a soft plastic covering (such as polyurethane having a durometer hardness of 80A available under the trade name Tecoflex) over-extruded over the wire core. The wires are embedded in the plastic covering to preclude accidental movement of the wires with respect to the wire core. Subsequently, an insulating layer of plastic is over-extruded over the soft core covering layer. This insulating layer form a hard outer layer. There is a need in the art for an improved system and method for manufacturing a lead body. In particular, there is a need in the art for a system and method for manufacturing a lead body that is capable of protecting and accurately placing electrically conducting conductors within the lead body during the manufacturing process. There is also a need in the art for a system and method for manufacturing a lead body that has a minimal diameter.

### SUMMARY OF THE INVENTION

The present invention is directed to a system and method for manufacturing a lead that includes a first layer unitary body that comprises a first plurality of conductors and a second layer that comprises a second plurality of conductors and extrusion material.

In one advantageous embodiment of the present invention, a first layer unitary body of a lead body assembly is formed by placing an inner layer of extrusion material on a mandrel. A first plurality of conductors is provided in which each conductor is coated with extrusion material. Each coated conductor is wrapped around the inner layer of extrusion material on the mandrel. An outer layer of extrusion material is then placed over the first plurality of conductors that are coated with extrusion material. Heat shrink tubing is then placed over the lead body assembly and the lead body assembly is heated to melt the extrusion material. The melted extrusion material is compressed around the plurality of conductors as the heat shrink tubing shrinks. The first layer unitary body assembly is then cooled to form a first layer unitary body and the heat shrink tubing is removed. The solidified extrusion material forms a protective wall that encapsulates the first plurality of conductors in the first layer unitary body.

In a first advantageous embodiment of the present invention, a lead body assembly is formed by preparing a first layer unitary body as previously described. An inner layer of extrusion material is placed on the first layer unitary body. A second plurality of conductors is provided in which each conductor is coated with extrusion material. Each coated conductor is wrapped around (or, alternatively, placed lengthwise on) the inner layer of extrusion material. An outer layer of extrusion material is then placed over the second plurality of conductors that are coated with extrusion material. Heat shrink tubing is then placed over the lead body assembly and the lead body assembly is heated to melt the extrusion material. The melted extrusion material is compressed around the first layer unitary body, the inner layer of extrusion material, the second plurality of conductors, and the outer layer of extrusion material as the heat shrink tubing shrinks. The lead body assembly is then cooled to form a lead body and the heat shrink tubing is removed. The solidified extrusion material forms a protective wall that encapsulates the first plurality of conductors and the second plurality of conductors. The lead body is then removed from the mandrel.

In a second advantageous embodiment of the present invention, a lead body assembly is formed by preparing a first layer unitary body as previously described. A second plurality of conductors is provided in which each conductor is coated with extrusion material. Each coated conductor is wrapped around (or, alternatively, placed lengthwise on) the first layer unitary body. An outer layer of extrusion material is then placed over the second plurality of conductors that are coated with extrusion material. Heat shrink tubing is then placed over the lead body assembly and the lead body assembly is heated to melt the extrusion material. The melted extrusion material is compressed around the first layer unitary body, the second plurality of conductors, and the outer layer of extrusion material as the heat shrink tubing shrinks. The lead body assembly is then cooled to form a lead body and the heat shrink tubing is removed. The solidified extrusion material forms a protective wall that encapsulates the first plurality of conductors and the second plurality of conductors. The lead body is then removed from the mandrel.

In a third advantageous embodiment of the present invention, a lead body assembly is formed by preparing a first layer unitary body as previously described. An inner layer of extrusion material is placed on the first layer unitary body. A second plurality of conductors is provided in which each conductor is coated with extrusion material. Each coated conductor is wrapped around (or, alternatively, placed lengthwise on) the inner layer of extrusion material. Heat shrink tubing is then placed over the lead body assembly and the lead body assembly is heated to melt the extrusion material. The melted extrusion material is compressed around the first layer unitary body, the inner layer of extrusion material, and the second plurality of conductors as the heat shrink tubing shrinks. The lead body assembly is then cooled to form a lead body and the heat shrink tubing is removed. The solidified extrusion material forms a protective wall that encapsulates the first plurality of conductors and the second plurality of conductors. The lead body is then removed from the mandrel.

In a fourth advantageous embodiment of the present invention, a lead body assembly is formed by preparing a first layer unitary body as previously described. A second plurality of conductors is provided in which each conductor is coated with extrusion material. Each coated conductor is wrapped around (or, alternatively, placed lengthwise on) the first layer unitary body. Heat shrink tubing is then placed over the lead body assembly and the lead body assembly is heated to melt the extrusion material. The melted extrusion material is compressed around the first layer unitary body and the second plurality of conductors as the heat shrink tubing shrinks. The lead body assembly is then cooled to form a lead body and the heat shrink tubing is removed. The solidified extrusion material forms a protective wall that encapsulates the first plurality of conductors and the second plurality of conductors. The lead body is then removed from the mandrel.

In a fifth advantageous embodiment of the present invention, a lead body assembly is formed by preparing a first layer unitary body as previously described. An inner layer of extrusion material is placed on the first layer unitary body. A second plurality of conductors is provided. Each conductor is wrapped around (or, alternatively, placed lengthwise on) the inner layer of extrusion material. An outer layer of extrusion material is then placed over the second plurality of conductors. Heat shrink tubing is then placed over the lead body assembly and the lead body assembly is heated to melt the extrusion material. The melted extrusion material is compressed around the first layer unitary body, the inner layer of extrusion material, the second plurality of conductors, and the outer layer of extrusion material as the heat shrink tubing shrinks. The lead body assembly is then cooled to form a lead body and the heat shrink tubing is removed. The solidified extrusion material forms a protective wall that encapsulates the first plurality of conductors and the second plurality of conductors. The lead body is then removed from the mandrel.

The foregoing has outlined rather broadly the features and technical advantages of the present invention so that those skilled in the art may better understand the detailed description of the invention that follows. Additional features s and advantages of the invention will be described hereinafter that form the subject of the claims of the invention. Those skilled in the art should appreciate that they may readily use the conception and the specific embodiment disclosed as a basis for modifying or designing other structures for carrying out the same purposes of the present invention. Those skilled in the art should also realize that such equivalent constructions do not depart from the spirit and scope of the invention in its broadest form.

### BRIEF DESCRIPTION OF THE DRAWINGS

For a more complete understanding of the present invention, and the advantages thereof, reference is now made to the following descriptions and the accompanying drawings, wherein like numbers designate like objects, and in which:

FIG. 1 illustrates a perspective view of a lead constructed in accordance with the present invention;

FIG. 2 illustrates a lead of the present invention connected to a stimulation source that includes an implantable pulse generator (IPG);

FIG. 3 illustrates a lead of the present invention connected to a stimulation source that includes a radio frequency receiver;

FIG. 4 illustrates a cross sectional view of an advantageous embodiment of a first layer unitary body assembly comprising an inner layer of extrusion material, a first plurality of conductors coated with a layer of extrusion material, and an outer layer of extrusion material;

FIG. 5 illustrates a cross sectional view of an advantageous embodiment of a first layer unitary body formed by subjecting the first layer unitary body assembly shown in FIG. 4 to melting and compression;

FIG. 6 illustrates a cross sectional view of a first embodiment of a lead body assembly of the present invention comprising a first layer unitary body as shown in FIG. 5 and a second layer comprising an inner layer of extrusion material, a second plurality of conductors coated with a layer of extrusion material and an outer layer of extrusion material;

FIG. 7 illustrates a cross sectional view of a first embodiment of the lead body of the present invention formed by subjecting the lead body assembly shown in FIG. 6 to melting and compression;

FIG. 8 illustrates a cross sectional view of a second embodiment of a lead body assembly of the present invention comprising a first layer unitary body as shown in FIG. 5 and a second layer comprising a second plurality of conductors coated with a layer of extrusion material and an outer layer of extrusion material;

FIG. 9 illustrates a cross sectional view of a second embodiment of the lead body of the present invention formed by subjecting the lead body assembly shown in FIG. 8 to melting and compression;

FIG. 10 illustrates a cross sectional view of a third embodiment of a lead body assembly of the present invention comprising a first layer unitary body as shown in FIG. 5 and a second layer comprising an inner layer of extrusion material and a second plurality of conductors coated with a layer of extrusion material;

FIG. 11 illustrates a cross sectional view of a third embodiment of the lead body of the present invention formed by subjecting the lead body assembly shown in FIG. 10 to melting and compression;

FIG. 12 illustrates a cross sectional view of a fourth embodiment of a lead body assembly of the present invention comprising a first layer unitary body as shown in FIG. 5 and a second layer comprising a second plurality of conductors coated with a layer of extrusion material;

FIG. 13 illustrates a cross sectional view of a fourth embodiment of the lead body of the present invention formed by subjecting the lead body assembly shown in FIG. 12 to melting and compression;

FIG. 14 illustrates a cross sectional view of a fifth embodiment of a lead body assembly of the present invention comprising a first layer unitary body as shown in FIG. 5 and a second layer comprising a second plurality of conductors and an outer layer of extrusion material;

FIG. 15 illustrates a cross sectional view of a fifth embodiment of the lead body of the present invention formed by subjecting the lead body assembly shown in FIG. 14 to melting and compression;

FIG. 16 is a flow diagram illustrating the steps of an advantageous embodiment of a method for making a first embodiment of the lead body of the present invention;

FIG. 17 is a flow diagram illustrating the steps of an advantageous embodiment of a method for making a second embodiment of the lead body of the present invention;

FIG. 18 is a flow diagram illustrating the steps of an advantageous embodiment of a method for making a third embodiment of the lead body of the present invention;

FIG. 19 is a flow diagram illustrating the steps of an advantageous embodiment of a method for making a fourth embodiment of the lead body of the present invention;

FIG. 20 is a flow diagram illustrating the steps of an advantageous embodiment of a method for making a fifth embodiment of the lead body of the present invention;

FIG. 21 illustrates a longitudinal cross sectional view of a first layer unitary body of the present invention showing heat shrink material attached at each end of the first layer unitary body;

FIG. 22 illustrates a longitudinal cross sectional view of one end of the lead body of the present invention showing the application of heat shrink material to the end of the lead body to separate the first and second plurality of conductors;

FIG. 23 illustrates a cross sectional view of one end of the lead body of the present invention where the lead body is covered with a portion of heat shrink material;

FIG. 24 illustrates a cross sectional view of one end of the lead body of the present invention at a point where the lead body is covered with heat shrink material and at a point where the first layer unitary body of the present invention is also covered with heat shrink material;

FIG. 25 illustrates a perspective side view of a mandrel with an exemplary conductor of a first plurality of conductors wound around the mandrel in an inner layer of conductors and an exemplary conductor of a second plurality of conductors wound around the mandrel in an outer layer of conductors; and

FIG. 26 illustrates a perspective side view of a mandrel with a first exemplary conductor along the axial length of the mandrel in a first direction in an inner layer of conductors and a second exemplary conductor along the axial length of the mandrel in a second direction in an outer layer of conductors.

### DETAILED DESCRIPTION OF THE INVENTION

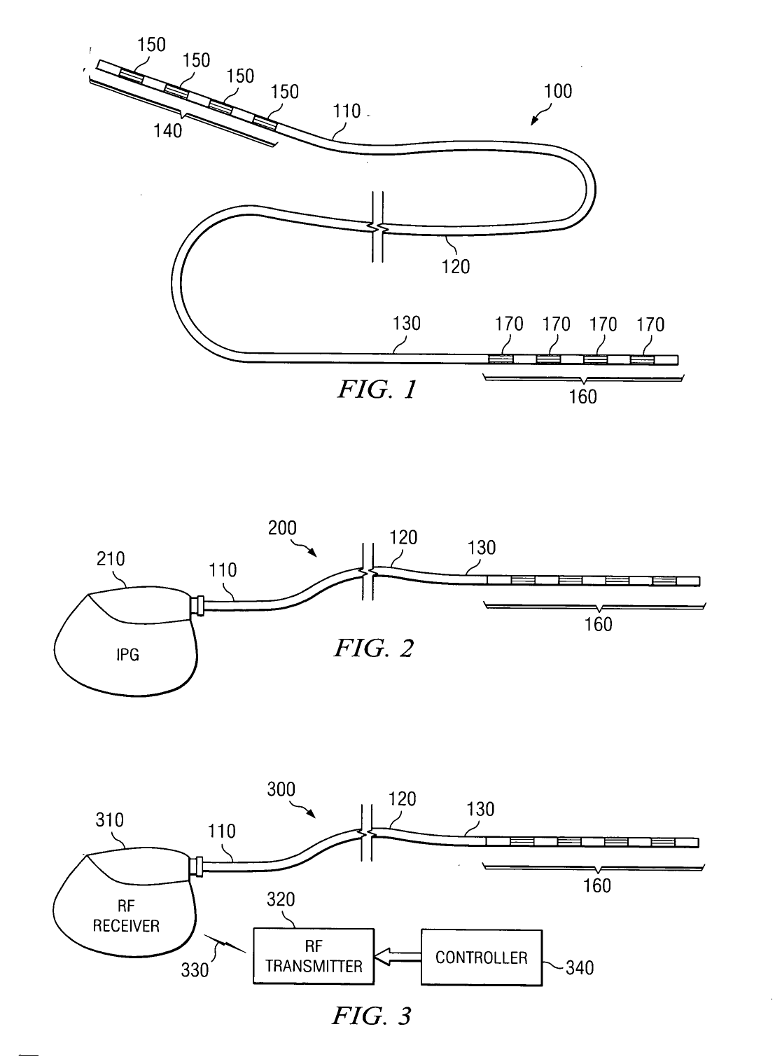

FIGS. 1 through 26, discussed below, and the various embodiments used to describe the principles of the present invention in this patent document are by way of illustration only and should not be construed in any way to limit the scope of the invention. Those skilled in the art will understand that the principles of the present invention may be implemented in any suitably modified medical lead.

FIG. 1 illustrates an advantageous embodiment of a lead 100 of the present invention. Lead 100 includes a flexible lead body 120 having a proximal end 110 and a distal end 130. Proximal end 110 of lead body 120 is coupled to an electrical contact 140. Distal end 130 of lead body 120 is coupled to electrode 160. Electrical contact 140 includes portions of lead body 120 and a plurality of contact electrodes 150 (also sometimes referred to as ring electrodes 150). Electrode 160 includes portions of lead body 120 and a plurality of band electrodes 170 (also sometimes referred to as ring electrodes 170). Although four contact electrodes 150 and four band electrodes 170 are shown in FIG. 1, it is understood that the present invention is not limited to the use of exactly four contact electrodes 150 or four band electrodes 170.

FIG. 2 and FIG. 3 illustrate different embodiments of a system (200, 300) for generating and applying a stimulus to a tissue or to a certain location of a body. In general terms, the system (200, 300) includes a stimulation or energy source (210, 310) and a lead 100 for application of the stimulus. The lead 100 shown in FIG. 2 and in FIG. 3 is the lead of the present invention.

FIG. 2 illustrates a lead 100 of the present invention connected to a stimulation source 210. The stimulation source 210 shown in FIG. 2 includes an implantable pulse generator (IPG). As is well known in the art, an implantable pulse generator (IPG) is capable of being implanted within a body (not shown) that is to receive electrical stimulation from the stimulation source 210. An exemplary implantable pulse generator (IPG) may be one manufactured by Advanced Neuromodulation Systems, Inc., such as the Genesis® System, part numbers 3604, 3608, 3609, and 3644. Reference numeral 200 refers to the system including the lead 100 and the stimulation source 210.

Electrical contact 140 is not visible in FIG. 2 because electrical contact 140 is situated within a receptacle (not shown) of stimulation source 210. Electrical contact 140 is electrically connected to a generator (not shown) of electrical signals within stimulation source 210. Stimulation source 210 generates and sends electrical signals via lead 100 to electrode 160. Electrode 160 is located at a stimulation site (not shown) within the body that is to receive electrical stimulation from the electrical signals. A stimulation site may be, for example, adjacent to one or more nerves in the central nervous system (e.g., spinal cord). The band electrodes 170 of electrode 160 conduct electrical signals from electrode 160 to the stimulation site. Stimulation source 210 is capable of controlling the electrical signals by varying signal parameters (e.g., intensity, duration, frequency) in response to control signals that are provided to stimulation source 210.

FIG. 3 illustrates a lead 100 of the present invention connected to a stimulation source 310. The stimulation source 310 shown in FIG. 3 includes a radio frequency (RF) receiver. As is well known in the art, a stimulation source 310 comprising a radio frequency (RF) receiver is capable of being implanted within the body (not shown) that is to receive electrical stimulation from the stimulation source 310. Exemplary RF receiver 310 may be those RF receivers manufactured by Advanced Neuromodulation Systems, Inc., such as the Renew® System, part numbers 3408 and 3416. Reference numeral 300 refers to the system including the lead 100 and the stimulation source 310. System 300 may also include the optional components 320 and 340 described below.

Electrical contact 140 is not visible in FIG. 3 because electrical contact 140 is situated within a receptacle (not shown) of stimulation source 310. Electrical contact 140 is electrically connected to a generator (not shown) of electrical signals within stimulation source 310. Stimulation source 310 generates and sends electrical signals via lead 100 to electrode 160. Electrode 160 is located at a stimulation site (not shown) within the body that is to receive electrical stimulation from the electrical signals. A stimulation site may be, for example, adjacent to one or more nerves in the central nervous system (e.g., spinal cord). The band electrodes 170 of electrode 160 conduct electrical signals from electrode 160 to the stimulation site. Stimulation source 310 is capable of controlling the electrical signals by varying signal parameters (e.g., intensity, duration, frequency) in response to control signals that are provided to stimulation source 310.

As shown in FIG. 3, the radio frequency (RF) receiver within stimulation source 310 is capable of receiving radio signals from a radio frequency (RF) transmitter 320. The radio signals are represented in FIG. 3 by radio link symbol 330. Radio frequency (RF) transmitter 320 and controller 340 are located outside of the body that is to receive electrical stimulation from stimulation source 310. A user of stimulation source 310 may use controller 340 to provide the control signals for the operation of stimulation source 310. Controller 340 provides the control signals to radio frequency (RF) transmitter 320. Radio frequency (RF) transmitter 320 transmits the control signals to the radio frequency (RF) receiver in stimulation source 310. Stimulation source 310 uses the control signals to vary the signal parameters of the electrical signals that are transmitted through electrical contact 140, lead body 120, and electrode 160 to the stimulation site. Exemplary RF transmitter 320 may be those RF transmitters manufactured by Advanced Neuromodulation Systems, Inc., such as the Renew® System, part numbers 3508 and 3516.

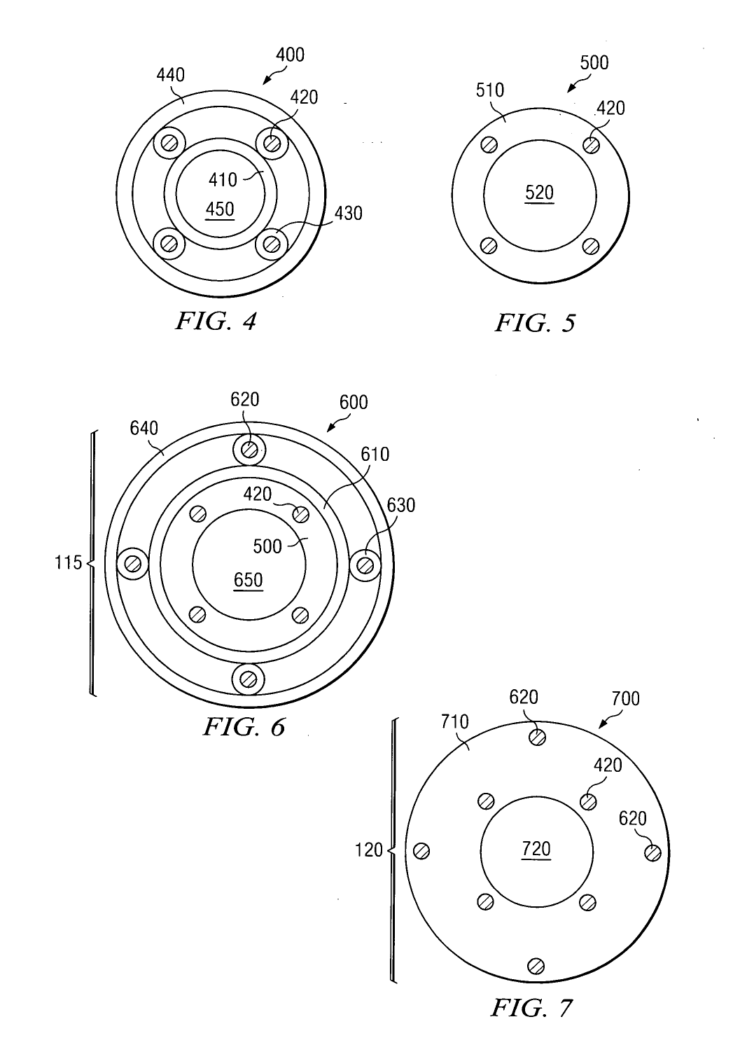

FIG. 4 illustrates a cross sectional view of an advantageous embodiment of a first layer unitary body assembly 400 of the lead body 120 of the present invention. The first layer unitary body assembly 400 of lead body 120 includes (1) an inner layer 410 of extrusion material, (2) a first plurality of conductors 420 in which each conductor 420 is coated with a layer of extrusion material 430, and (3) an outer layer 440 of extrusion material. A lumen 450 is formed by the inner wall of inner layer 410.

An advantageous embodiment of a method for making a first layer unitary body 500 of lead body 120 (shown in FIG. 5) will now be described. An inner layer 410 of extrusion material is placed on a cylindrically shaped mandrel (not shown). After the first layer unitary body assembly 400 is removed from the mandrel, the space formerly occupied by the mandrel will form lumen 450 within inner layer 410. Each conductor 420 of the first plurality of conductors 420 is coated with a layer 430 of the same extrusion material that is used to form inner layer 410. Alternatively, the extrusion material used to form layer 430 may not be the same type of extrusion material that is used to form inner layer 410. Each conductor 420 of the first plurality of conductors 420 is wrapped around (i.e., coiled around) the inner layer 410 of extrusion material. The layer 430 of extrusion material around each conductor 420 ensures that the conductors 420 are uniformly spaced. An outer layer 440 of extrusion material is placed over the plurality of conductors 420. The outer layer 440 of extrusion material forms an external coating over the first plurality of conductors 420 as shown in FIG. 4.

In an alternative embodiment of the method of the present invention, each conductor 420 of the first plurality of conductors 420 is not coiled around the inner layer 410 of extrusion material, but instead is placed lengthwise along the axial length of inner layer 410. An outer layer 440 of extrusion material is placed over the first plurality of conductors 420 in the same manner as in the case of the coiled conductors 420.

The extrusion material is formed of an insulating material typically selected based upon biocompatibility, biostability and durability for the particular application. The extrusion material may be silicone, polyurethane, polyethylene, polyimide, polyvinylchloride, PTFT, EFTE, or other suitable materials known to those skilled in the art. Alloys or blends of these materials may also be formulated to control the relative flexibility, torqueability, and pushability of the lead body 120. Depending on the particular application, the diameter of the lead body 120 may be any size, though a smaller size is more desirable for neurological, cardiac pacing and myocardial mapping/ablation leads and neuromodulation and stimulation leads.

The conductors may take the form of solid conductors, drawn-filled-tube (DFT), drawn-brazed-strand (DBS), stranded conductors or cables, ribbons conductors, or other forms known or recognized to those skilled in the art. The composition of the conductors may include aluminum, stainless steel, MP35N, platinum, gold, silver, copper, vanadium, alloys, or other conductive materials or metals known to those of ordinary skill in the art. The number, size, cross-sectional shape, and composition of the conductors will depend on the particular application for the lead body 120.

As previously mentioned, the conductors 420 may be configured along the first layer unitary body assembly 400 in a straight orientation or cylindrically or helically wound around the lumen 450 at the center of the first layer unitary body assembly 400. The conductors 420 are typically insulated from the lumen 450, and from each other, and from the external surface of the first layer unitary body assembly 400 by the extrusion material. As also previously mentioned, the extrusion material may be of single composition, or of multiple layers of the same or different materials.

First layer unitary body assembly 400 is then covered with heat shrink tubing (not shown) and heat is applied. The heat melts the layers (410, 430 and 440) of extrusion material and the melted extrusion material flows together to form an integral body. The heat shrink tubing holds and compresses the extrusion material and the conductors that are located within the extrusion material to create a first layer unitary body 500 as shown in FIG. 5. The conductors 420 in first layer unitary body 500 may each be centered within the wall 510 of the first layer unitary body 500. Wall 510 is formed from the layers that include the layers (410, 430 and 440) of extrusion material shown in FIG. 4. The first layer unitary body 500 is cooled and the heat shrink tubing removed. Lumen 520 is formed when the first layer unitary body 500 is removed from the mandrel (not shown). There may be some release of coiled tension in the conductors 420 when the heat shrink tubing is removed.

The present invention provides a layer 430 of extrusion material around each conductor 420. This protective layer 430 of extrusion material provides an electrical barrier between each of the conductors 420. This process also provides a significant improvement over the prior art method that uses a mechanical comb in the winders to try to keep the conductors 420 separate. The protective layer 430 of extrusion material also allows the present invention to create leads that are smaller and thinner than prior art leads. In general, the inventive lead body diameter will be smaller than 34 French and can be smaller than 9 French. This holds true for all the embodiments described below.

The method of the present invention provides several advantages over prior art methods. Advantages of the method of the present invention include: (1) more accurate conductor placement during the process of coiling the conductor around a mandrel, (2) more accurate conductor pitches, (3) improved pitch consistency, (4) more conductor protection during the process of coiling the conductor around the mandrel, and (5) precise centering of the conductors within the resulting unitary body.

Importantly, the apparatus and method of the present invention makes possible the construction of lead bodies that have a smaller diameter than prior art lead bodies. That is, the lead bodies of the present invention may be made thinner than prior art lead bodies. The cylindrically symmetrical embodiment of the lead body 120 of the invention can also better withstand lateral stretching than prior art lead bodies.

The first layer unitary body assembly 400 has been described as having cylindrical symmetry. It is noted that other types of geometrical cross-sectional shapes (e.g., rectangular) could be used if a different shape is desired for a particular application.

The first layer unitary body assembly 400 of lead body 120 has been shown as having four conductors 420. The use of four conductors 420 is merely an example. It is understood that more than four conductors 420 and fewer than four conductors 420 may be used. In one advantageous embodiment eight conductors 420 are used in the first layer unitary body assembly 400.

The method for forming first layer unitary body 500 of lead body 120 that has been described is not the only method that may be used. Other methods for forming first layer unitary body 500 are described in co-pending U.S. patent application Ser. No. [Attorney Docket No. 03-002], and are incorporated herein by reference for all purposes as if fully set forth herein.

After the first layer unitary body 500 of lead body 120 has been formed, additional conductors and extrusion material are applied over first layer unitary body 500 to form a second layer of lead body 120.

FIG. 6 illustrates a cross sectional view of a first embodiment of a lead body assembly 115 of the present invention. Lead body assembly 115 includes (1) the first layer unitary body 500 containing conductors 420, (2) an inner layer 610 of extrusion material on the first layer unitary body 500, (3) a second plurality of conductors 620 in which each conductor 620 is coated with a layer of extrusion material 630, and (4) an outer layer 640 of extrusion material. A lumen 650 is formed by the inner wall of first layer unitary body 500. The portions of the first embodiment of lead body assembly 115 shown in FIG. 6 are collectively referred to with reference numeral 600.

An advantageous embodiment of a method for making the first embodiment of lead body 120 (shown in FIG. 7) will now be described. An inner layer 610 of extrusion material is placed on a cylindrically shaped first layer unitary body 500 that has been formed as previously described. After the mandrel is removed from first layer unitary body 500, the space formerly occupied by the mandrel in first layer unitary body 500 will form lumen 650. Each conductor 620 of the second plurality of conductors 620 is coated with a layer 630 of the same extrusion material that is used to form inner layer 610. Alternatively, the extrusion material used to form layer 630 may not be the same type of extrusion material that is used to form inner layer 610. Each conductor 620 of the second plurality of conductors 620 is cylindrically wrapped around (i.e., coiled around) the inner layer 610 of extrusion material. The layer 630 of extrusion material around each conductor 620 ensures that the conductors 620 are uniformly spaced. An outer layer 640 of extrusion material is placed over the second plurality of conductors 620. The outer layer 640 of extrusion material forms an external coating over the second plurality of conductors 620 as shown in FIG. 6.

In one advantageous embodiment of lead body 120, the conductors 420 are wrapped within first layer unitary body 500 in a first direction and the conductors 620 are wrapped around inner layer 610 in the same first direction. In another advantageous embodiment of lead body 120, the conductors 420 are wrapped within first layer unitary body 500 in a first direction and the conductors 620 are wrapped around inner layer 610 in an opposite second direction. For additional description of this feature, refer to U.S. Pat. No. [Attorney Docket Number 03-003A] filed concurrently herewith, entitled “System and Method for Providing A Medical Lead Body Having Conductors That Are Wound in Opposite Directions,” incorporated herein by reference for all purposes.

In an alternative advantageous embodiment of lead body 120, the conductors 620 may be placed along the axial length of inner layer 610 of lead body 120 in a straight orientation. In this embodiment, each conductor 620 is not coiled around the inner layer 610 of lead body 120, but instead is placed lengthwise along the axial length of inner layer 610. An outer layer 640 of extrusion material is placed over the plurality of conductors 620 in the same manner as in the case of the coiled conductors 620.

The conductors 620 are typically insulated from the lumen 650, and from each other, and from the external surface of the lead body 120 by the extrusion material. As previously mentioned, the extrusion material may be of single composition, or of multiple layers of the same or different materials.

The combined portions 600 of lead body assembly 115 are then covered with heat shrink tubing (not shown) and heat is applied. The heat melts the layers (610, 630 and 640) of extrusion material and the melted extrusion material flows together to form an integral body. The heat shrink tubing holds and compresses the melted extrusion material around the conductors that are located within the extrusion material to create a unitary body lead 700 as shown in FIG. 7.

In FIG. 7, the conductors 420 in first layer unitary body 500 and the conductors 620 are each encapsulated within a unitary or uniform wall 710 of the unitary body lead 700. These conductors are contained in the unitary core that comprises the unitary or unified wall 710, lumen 720 and conductors 420 and 620. Unitary wall 710 is formed from the materials included in the first layer unitary body 500 and the layers (610, 630 and 640) of extrusion material shown in FIG. 6. The unitary body lead 700 is cooled and the heat shrink tubing removed. Lumen 720 is formed when the unitary body lead 700 is removed from the mandrel (not shown). There may be some release of coiled tension in the conductors, 420 and 620, when the heat shrink tubing is removed.

The present invention provides a layer 630 of extrusion material around each conductor 620. This protective layer 630 of extrusion material provides an electrical barrier between each of the conductors 620. This process also provides a significant improvement over the prior art method that uses a mechanical comb in the winders to try to keep the conductors 620 separate. As previously mentioned, the layer 630 of extrusion material around each conductor 620 also ensures that the conductors 620 are uniformly spaced within wall 710.

The lead body assembly 115 shown in FIG. 6 has been described as having cylindrical symmetry. It is noted that other types of geometrical cross-sectional shapes (e.g., rectangular) could be used if a different shape is desired for a particular application.

The lead body assembly 115 shown in FIG. 6 has been shown as having four conductors 420 and four conductors 620. The use of four conductors 420 and four conductors 620 is merely an example. It is understood that more than four conductors 420 and more than four conductors 620 may be used. It is also understood that fewer than four conductors 420 and fewer than four conductors 620 may be used. In one advantageous embodiment eight conductors 420 and eight conductors 620 are used in lead body 120.

As described above, once formed, there is no need to have a separate or secondary electrical insulation material (separate from the extrusion material that forms unitary wall 710) surrounding the conductors as in the prior art. This is because the unitary construction of wall 710 acts as the electrical insulation material and forms the unitary core or wall of the unitary body. This is true for embodiments of this invention including those described below.

Wall 710 is formed from the layers that include the layers (610, 630 and 640) of extrusion material shown in FIG. 6. As known, the various extrusion materials may be of a like kind or may be formulated using different materials such that when formed as a unitary body, the lead body will have a desired consistence, flexibility, electrically conductive properties, or other such functionality as may be desired. This holds true for all embodiments of the invention described below.

In the embodiment described above, the unitary body lead 500 is cooled and the heat shrink tubing removed. Lumen 720 is formed when the unitary body lead 700 is removed from the mandrel (not shown). There may be some release of coiled tension in the conductors 620 when the heat shrink tubing is removed. This holds true for all embodiments of the invention described below.

While the previous paragraph describe one embodiment of forming the unitary body, those skilled in the art will recognize that other like methods may be used. For example, some of the other possible ways of forming the lead without heat shrink could be: a single hot die, successively smaller dies wherein the dies are used to draw the product to a final outside diameter. Other methods could be a compression mold or hot die drawing or other methods familiar to those in the arts. In fact as those skilled will understand, any heating method that results in the wires becoming imbedded in a homogenous plastic or unitary body may be used. This holds true for all embodiments of the invention described below.

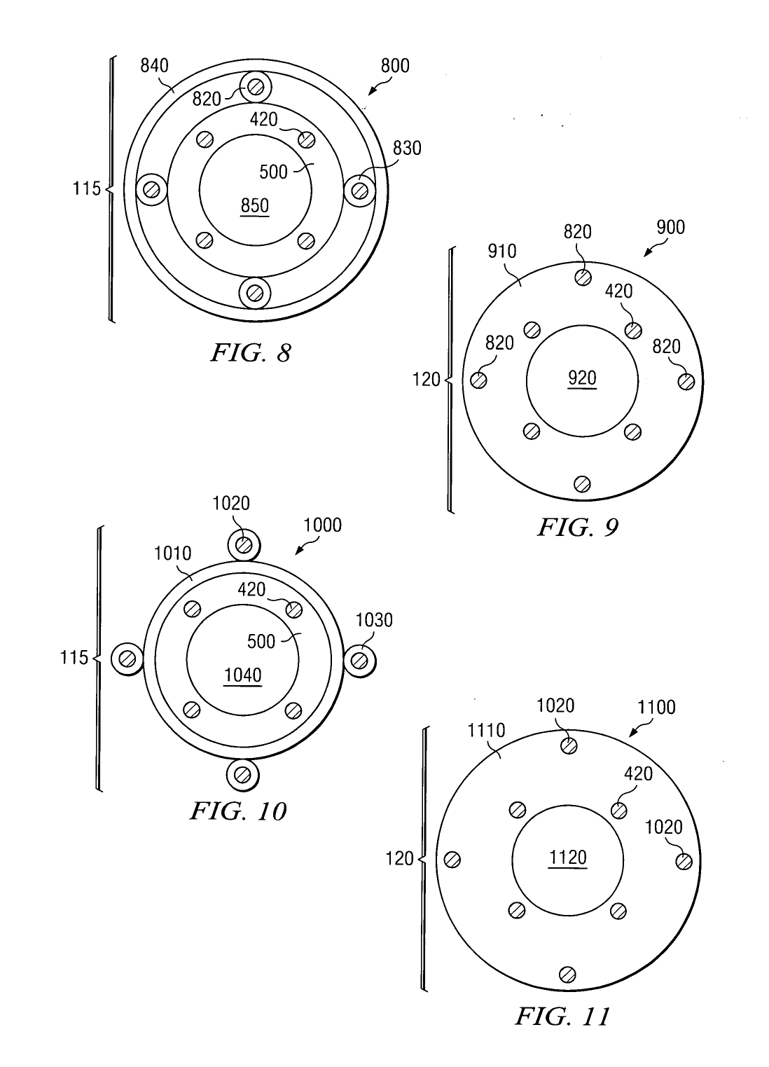

FIG. 8 illustrates a cross sectional view of a second embodiment of a lead body assembly 115 of the present invention. Lead body assembly 115 includes (1) the first layer unitary body 500 containing conductors 420, (2) a second plurality of conductors 820 on the first layer unitary body 500, in which each conductor 820 is coated with a layer of extrusion material 830, and (3) an outer layer 840 of extrusion material. A lumen 850 is formed by the inner wall of first layer unitary body 500. The portions of the second embodiment of lead body assembly 115 shown in FIG. 8 are collectively referred to with reference numeral 800.

An advantageous embodiment of a method for making the second embodiment of lead body 120 (shown in FIG. 9) will now be described. A second plurality of conductors 820 is provided in which each conductor 820 is coated with a layer 830 of extrusion material. Each conductor 820 of the second plurality of conductors 820 is coated with a layer 830 of the same extrusion material that is used to form first layer unitary body 500. Alternatively, the extrusion material used to form layer 830 may not be the same type of extrusion material that is used to form first layer unitary body 500. Each conductor 820 of the second plurality of conductors 820 is cylindrically wrapped around (i.e., coiled around) first layer unitary body 500 that has been formed as previously described. The layer of extrusion material 830 around each conductor 820 ensures that the conductors 820 are uniformly spaced. An outer layer 840 of extrusion material is placed over the second plurality of conductors 820. The outer layer 840 of extrusion material forms an external coating over the second plurality of conductors 820 as shown in FIG. 8.

In one advantageous embodiment of lead body 120, the conductors 420 are wrapped within first layer unitary body 500 in a first direction and the conductors 820 are wrapped around first layer unitary body 500 in the same first direction. In another advantageous embodiment of lead body 120, the conductors 420 are wrapped within first layer unitary body 500 in a first direction and the conductors 820 are wrapped around first layer unitary body 500 in an opposite second direction.

In another advantageous embodiment of lead body 120, the conductors 820 may be placed along the axial length of first layer unitary body 500 of lead body 120 in a straight orientation. In this embodiment, each conductor 820 is not coiled around the first layer unitary body 500, but instead is placed lengthwise along the axial length of first layer unitary body 500. An outer layer 840 of extrusion material is placed over the plurality of conductors 820 in the same manner as in the case of the coiled conductors 820.

The conductors 820 are typically insulated from the lumen 850, and from each other, and from the external surface of the lead body 120 by the extrusion material. As previously mentioned, the extrusion material may be of single composition, or of multiple layers of the same or different materials.

The combined portions 800 of lead body assembly 115 are then covered with heat shrink tubing (not shown) and heat is applied. The heat melts the layers (830 and 840) of extrusion material and the melted extrusion material flows together to form an integral body. The heat shrink tubing holds and compresses the melted extrusion material around the conductors that are located within the extrusion material to create a unitary body lead 900 as shown in FIG. 9. The conductors 420 in first layer unitary body 500 and the conductors 820 are each encapsulated within the wall 910 of the unitary body lead 900. Wall 910 is formed from the materials that are included in the first layer unitary body 500 and the layers (830 and 840) of extrusion material shown in FIG. 8. The unitary body lead 900 is cooled and the heat shrink tubing removed. Lumen 920 is formed when the unitary body lead 900 is removed from the mandrel (not shown). There may be some release of coiled tension in the conductors when the heat shrink tubing is removed.

The present invention provides a layer 830 of extrusion material around each conductor 820. This protective layer 830 of extrusion material provides an electrical barrier between each of the conductors 820. This process also provides a significant improvement over the prior art method that uses a mechanical comb in the winders to try to keep the conductors 820 separate. The protective layer 830 of extrusion material also allows the present invention to create leads that are smaller and thinner than prior art leads.

The lead body assembly 115 shown in FIG. 8 has been described as having cylindrical symmetry. It is noted that other types of geometrical cross-sectional shapes (e.g., rectangular) could be used if a different shape is desired for a particular application.

The lead body assembly 115 shown in FIG. 8 has been shown as having four conductors 420 and four conductors 820. The use of four conductors 420 and four conductors 820 is merely an example. It is understood that more than four conductors 420 and more than four conductors 820 may be used. It is also understood that fewer than four conductors 420 and fewer than four conductors 820 may be used. In one advantageous embodiment eight conductors 420 and eight conductors 820 are used in lead body 120.

FIG. 10 illustrates a cross sectional view of a third embodiment of a lead body assembly 115 of the present invention. Lead body assembly 115 includes (1) the first layer unitary body 500 containing conductors 420, (2) an inner layer 1010 of extrusion material on the first layer unitary body 500, and (3) a second plurality of conductors 1020 on the inner layer 1010, in which each conductor 1020 is coated with a layer of extrusion material 1030. A lumen 1040 is formed by the inner wall of first layer unitary body 500. The portions of the third embodiment of lead body assembly 115 shown in FIG. 10 are collectively referred to with reference numeral 1000.

An advantageous embodiment of a method for making the third embodiment of lead body 120 (shown in FIG. 11) will now be described. A second plurality of conductors 1020 is provided in which each conductor 1020 is coated with a layer 1030 of extrusion material. Each conductor 1020 of the second plurality of conductors 1020 is coated with a layer 1030 of the same extrusion material that is used to form first layer unitary body 500. Alternatively, the extrusion material used to form layer 1030 may not be the same type of extrusion material that is used to form first layer unitary body 500. Each conductor 1020 of s the second plurality of conductors 1020 is cylindrically wrapped around (i.e., coiled around) the inner layer 1010 placed on the first layer unitary body 500 that has been formed as previously described. The layer 1030 of extrusion material around each conductor 1020 ensures that the conductors 1020 are uniformly spaced.

In one advantageous embodiment of lead body 120, the conductors 420 are wrapped within first layer unitary body 500 in a first direction and the conductors 1020 are wrapped around inner layer 1010 in the same first direction. In another advantageous embodiment of lead body 120, the conductors 420 are wrapped within first layer unitary body 500 in a first direction and the conductors 1020 are wrapped around inner layer 1010 in an opposite second direction.

In another advantageous embodiment of lead body 120, the conductors 1020 may be placed along the axial length of inner layer 1010 of lead body 120 in a straight orientation. In this embodiment, each conductor 1020 is not coiled around the inner layer 1010, but instead is placed lengthwise along the axial length of inner layer 1010.

The conductors 1020 are typically insulated from the lumen 1040, and from each other, and from the external surface of the lead body 120 by the extrusion material. As previously mentioned, the extrusion material may be of single composition, or of multiple layers of the same or different materials.

The combined portions 1000 of lead body assembly 115 are then covered with heat shrink tubing (not shown) and heat is applied. The heat melts the layers (1010 and 1030) of extrusion material and the melted extrusion material flows together to form an integral body. The heat shrink tubing holds and compresses the melted extrusion material around the conductors that are located within the extrusion material to create a unitary body lead 1100 as shown in FIG. 11. The conductors 420 in first layer unitary body 500 and the conductors 1020 are each encapsulated within the wall 1110 of the unitary body lead 1100. Wall 1110 is formed from the materials that are included in the first layer unitary body 500 and the layers (1010 and 1030) of extrusion material shown in FIG. 10. The unitary body lead 1100 is cooled and the heat shrink tubing removed. Lumen 1120 is formed when the unitary body lead 1100 is removed from the mandrel (not shown). There may be some release of coiled tension in the conductors when the heat shrink tubing is removed.

The present invention provides a layer 1030 of extrusion material around each conductor 1020. This protective layer 1030 of extrusion material provides an electrical barrier between each of the conductors 1020. This process also provides a significant improvement over the prior art method that uses a mechanical comb in the winders to try to keep the conductors 1020 separate. The protective layer 1030 of extrusion material also allows the present invention to create leads that are smaller and thinner than prior art leads.

The lead body assembly 115 shown in FIG. 10 has been described as having cylindrical symmetry. It is noted that other types of geometrical cross-sectional shapes (e.g., rectangular) could be used if a different shape is desired for a particular application.

The lead body assembly 115 shown in FIG. 10 has been shown as having four conductors 420 and four conductors 1020. The use of four conductors 420 and four conductors 1020 is merely an example. It is understood that more than four conductors 420 and more than four conductors 1020 may be used. It is also understood that fewer than four conductors 420 and fewer than four conductors 1020 may be used. In one advantageous embodiment eight conductors 420 and eight conductors 1020 are used in lead body 120.

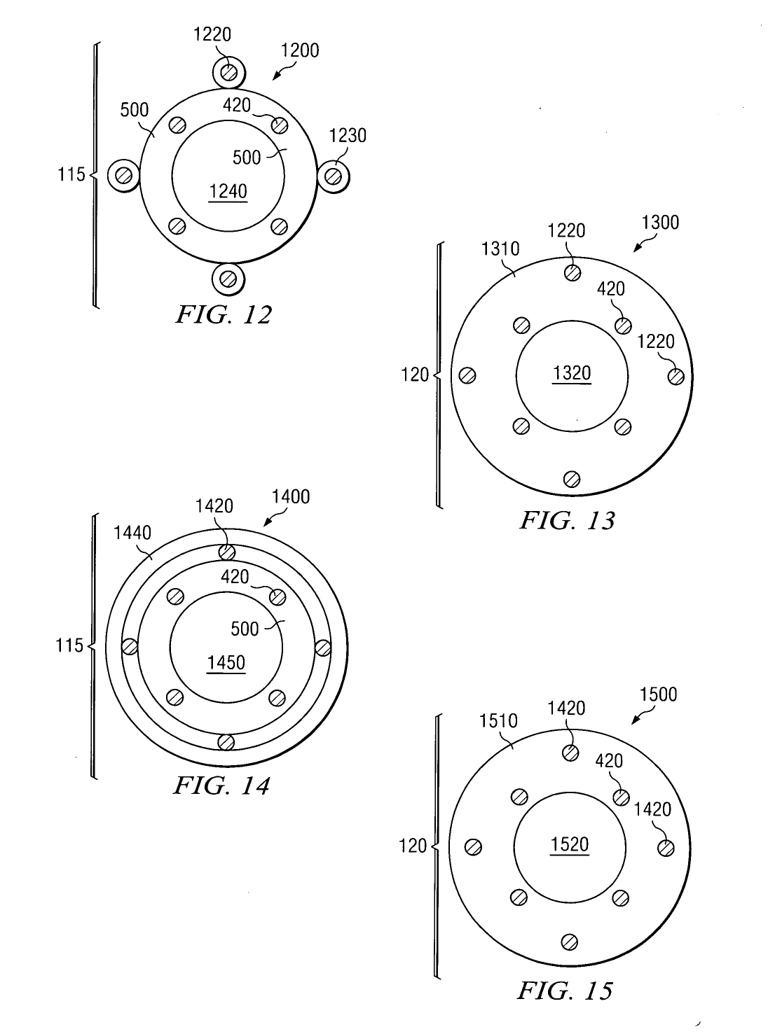

FIG. 12 illustrates a cross sectional view of a fourth embodiment of a lead body assembly 115 of the present invention. Lead body assembly 115 includes (1) the first layer unitary body 500 containing conductors 420, and (2) a second plurality of conductors 1220 on the first layer unitary body 500, in which each conductor 1220 is coated with a layer of extrusion material 1230. A lumen 1240 is formed by the inner wall of first layer unitary body 500. The portions of the fourth embodiment of lead body assembly 115 shown in FIG. 12 are collectively referred to with reference numeral 1200.

An advantageous embodiment of a method for making the fourth embodiment of lead body 120 (shown in FIG. 13) will now be described. A second plurality of conductors 1220 is provided in which each conductor 1220 is coated with a layer 1230 of extrusion material. Each conductor 1220 of the second plurality of conductors 1220 is coated with a layer 1230 of the same extrusion material that is used to form first layer unitary body 500. Alternatively, the extrusion material used to form layer 1230 may not be the same type of extrusion material that is used to form first layer unitary body 500. Each conductor 1220 of the second plurality of conductors 1220 is cylindrically wrapped around (i.e., coiled around) first layer unitary body 500 that has been formed as previously described.

In one advantageous embodiment of lead body 120, the conductors 420 are wrapped within first layer unitary body 500 in a first direction and the conductors 1220 are wrapped around first layer unitary body 500 in the same first direction. In another advantageous embodiment of lead body 120, the conductors 420 are wrapped within first layer unitary body 500 in a first direction and the conductors 1220 are wrapped around first layer unitary body 500 in an opposite second direction.

In another advantageous embodiment of lead body 120, the conductors 1220 may be placed along the axial length of first layer unitary body 500 of lead body 120 in a straight orientation. In this embodiment, each conductor 1220 is not coiled around the first layer unitary body 500, but instead is placed lengthwise along the axial length of first layer unitary body 500.

The conductors 1220 are typically insulated from the lumen 1240, and from each other, and from the external surface of the lead body 120 by the extrusion material. As previously mentioned, the extrusion material may be of single composition, or of multiple layers of the same or different materials.

The combined portions 1200 of lead body assembly 115 are then covered with heat shrink tubing (not shown) and heat is applied. The heat melts the layers 1230 of extrusion material and the extrusion material of first layer unitary body 500. The melted extrusion material flows together to form an integral body. The heat shrink tubing holds and compresses the melted extrusion material around the conductors that are located within the extrusion material to create a unitary body lead 1300 as shown in FIG. 13. The conductors 420 in first layer unitary body 500 and the conductors 1220 are each encapsulated within the wall 1310 of the unitary body lead 1300. Wall 1310 is formed from the layers that are included in the first layer unitary body 500 and the layers 1230 of extrusion material shown in FIG. 12. The unitary body lead 1300 is cooled and the heat shrink tubing removed. Lumen 1320 is formed when the unitary body lead 1300 is removed from the mandrel (not shown). There may be some release of coiled tension in the conductors when the heat shrink tubing is removed.

The present invention provides a layer 1230 of extrusion material around each conductor 1220. This protective layer 1230 of extrusion material provides an electrical barrier between each of the conductors 1220. This process also provides a significant improvement over the prior art method that uses a mechanical comb in the winders to try to keep the conductors 1220 separate. The protective layer 1230 of extrusion material also allows the present invention to create leads that are smaller and thinner than prior art leads.

The lead body assembly 115 shown in FIG. 12 has been described as having cylindrical symmetry. It is noted that other types of geometrical cross-sectional shapes (e.g., rectangular) could be used if a different shape is desired for a particular application.

The lead body assembly 115 shown in FIG. 12 has been shown as having four conductors 420 and four conductors 1220. The use of four conductors 420 and four conductors 1220 is merely an example. It is understood that more than four conductors 420 and more than four conductors 1220 may be used. It is also understood that fewer than four conductors 420 and fewer than four conductors 1220 may be used. In one advantageous embodiment eight conductors 420 and eight conductors 1220 are used in lead body 120.

FIG. 14 illustrates a cross sectional view of a fifth embodiment of a lead body assembly 115 of the present invention. Lead body assembly 115 includes (1) the first layer unitary body 500 containing conductors 420, (2) a second plurality of conductors 1420 on the first layer unitary body 500, and (3) an outer layer 1440 of extrusion material. A lumen 1450 is formed by the inner wall of first layer unitary body 500. The portions of the fifth embodiment of lead body assembly 115 shown in FIG. 14 are collectively referred to with reference numeral 1400.

An advantageous embodiment of a method for making the fifth embodiment of lead body 120 (shown in FIG. 15) will now be described. A second plurality of conductors 1420 is provided. Each conductor 1420 of the second plurality of conductors 1420 is cylindrically wrapped around (i.e. coiled around) first layer unitary body 500 that has been formed as previously described. An outer layer 1440 of extrusion material is placed over the second plurality of conductors 1420. The outer layer 1440 of extrusion material forms an external coating over the second plurality of conductors 1420 as shown in FIG. 14.

In one advantageous embodiment of lead body 120, the conductors 420 are wrapped within first layer unitary body 500 in a first direction and the conductors 1420 are wrapped around first layer unitary body 500 in the same first direction. In another advantageous embodiment of lead body 120, the conductors 420 are wrapped within first layer unitary body 500 in a first direction and the conductors 1420 are wrapped around first layer unitary body 500 in an opposite second direction.

In another advantageous embodiment of lead body 120, the conductors 1420 may be placed along the length of first layer unitary body 500 of lead body 120 in a straight orientation. In this embodiment, each conductor 1420 is not coiled around the first layer unitary body 500 of lead body 120, but instead is placed lengthwise along the axial length of first layer unitary body 500. An outer layer 1440 of extrusion material is placed over the plurality of conductors 1420 in the same manner as in the case of the coiled conductors 1420.

The combined portions 1400 of lead body assembly 115 are then covered with heat shrink tubing (not shown) and heat is applied. The heat melts the outer layer 1440 of extrusion material and the extrusion material of first layer unitary body 500. The melted extrusion material flows together to form an integral body. The heat shrink tubing holds and compresses the melted extrusion material around the conductors that are located within the extrusion material to create a unitary body lead 1500 as shown in FIG. 15. The conductors 420 in first layer unitary body 500 and the conductors 1420 are each encapsulated within the wall 1510 of the unitary body lead 1500. Wall 1510 is formed from the materials that are included in the first layer unitary body 500 and the layer 1440 of extrusion material shown in FIG. 14. The unitary body lead 1500 is cooled and the heat shrink tubing removed. Lumen 1520 is formed when the unitary body lead 1500 is removed from the mandrel (not shown). There may be some release of coiled tension in the conductors when the heat shrink tubing is removed.

The lead body assembly 115 shown in FIG. 14 has been described as having cylindrical symmetry. It is noted that other types of geometrical cross-sectional shapes (e.g., rectangular) could be used if a different shape is desired for a particular application.

The lead body assembly 115 shown in FIG. 14 has been shown as having four conductors 420 and four conductors 1420. The use of four conductors 420 and four conductors 1420 is merely an example. It is understood that more than four conductors 420 and more than four conductors 1420 may be used. It is also understood that fewer than four conductors 420 and fewer than four conductors 1420 may be used. In one advantageous embodiment eight conductors 420 and eight conductors 1420 are used in lead body 120.

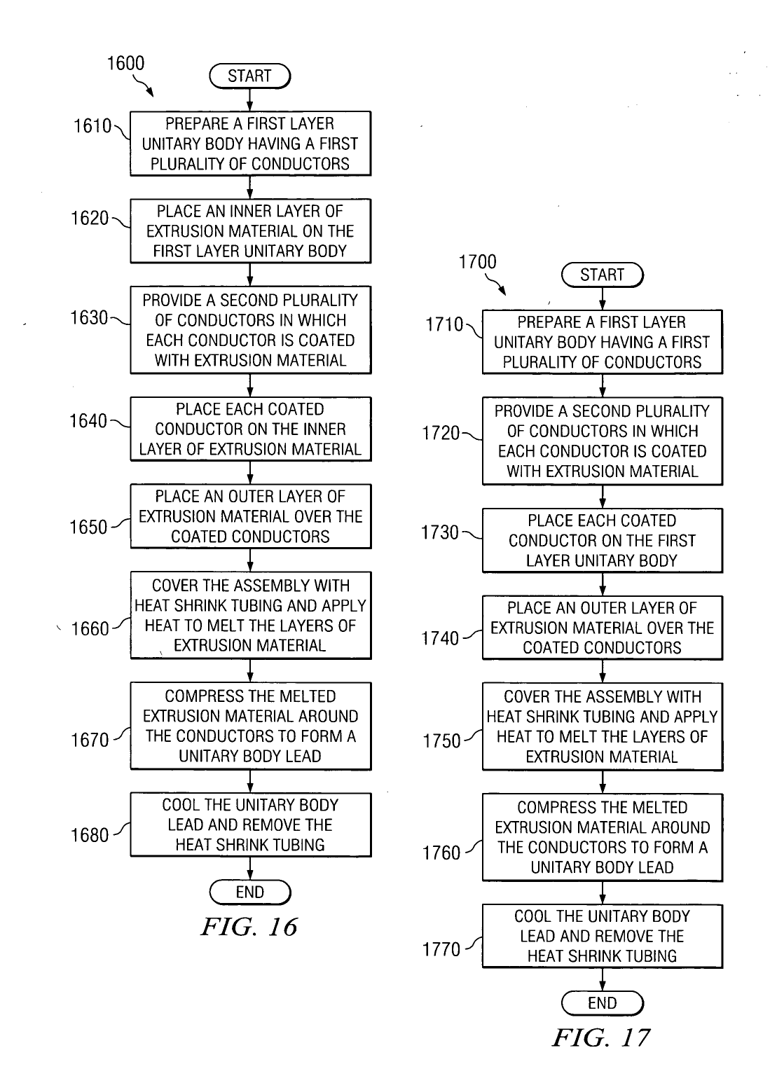

FIG. 16 illustrates a flow chart depicting the steps of one advantageous embodiment of the process of the present invention for making a first embodiment of lead body 120. The steps of the method are collectively referred to with reference numeral 1600.

A first body unitary layer 500 is prepared having a first plurality of conductors 420 (step 1610). An inner layer of extrusion material is placed over the first layer unitary body 500 (step 1620). A second plurality of conductors is provided in which each conductor is coated with extrusion material (step 1630). Each coated conductor is then wrapped around (or placed on) the inner layer of extrusion material (step 1640). An outer layer of extrusion material is then placed over the second plurality of coated conductors on the inner layer (step 1650).

The assembly of the first layer unitary body, the inner layer, the coated conductors, and the outer layer is then covered with heat shrink tubing and heat is applied to melt the layers of extrusion material (step 1660). The heat shrink tubing compresses the extrusion material around the conductors to form a unitary body lead (step 1670). The unitary body lead is then cooled and the heat shrink tubing is removed (step 1680).

FIG. 17 illustrates a flow chart depicting the steps of an advantageous embodiment of the method of the present invention for making a second embodiment of lead body 120. The steps of the method are collectively referred to with reference numeral 1700.

A first body unitary layer 500 is prepared having a first plurality of conductors 420 (step 1710). A second plurality of conductors is provided in which each conductor is coated with extrusion material (step 1720). Each coated conductor is then wrapped around (or placed on) the first layer unitary body 500 (step 1730). An outer layer of extrusion material is then placed over the second plurality of coated conductors (step 1740).

The assembly of the first layer unitary body and the coated conductors and the outer layer is then covered with heat shrink tubing and heat is applied to melt the layers of extrusion material (step 1750). The heat shrink tubing compresses the extrusion material around the conductors to form a unitary body lead (step 1760). The unitary body lead is then cooled and the heat shrink tubing is removed (step 1770).

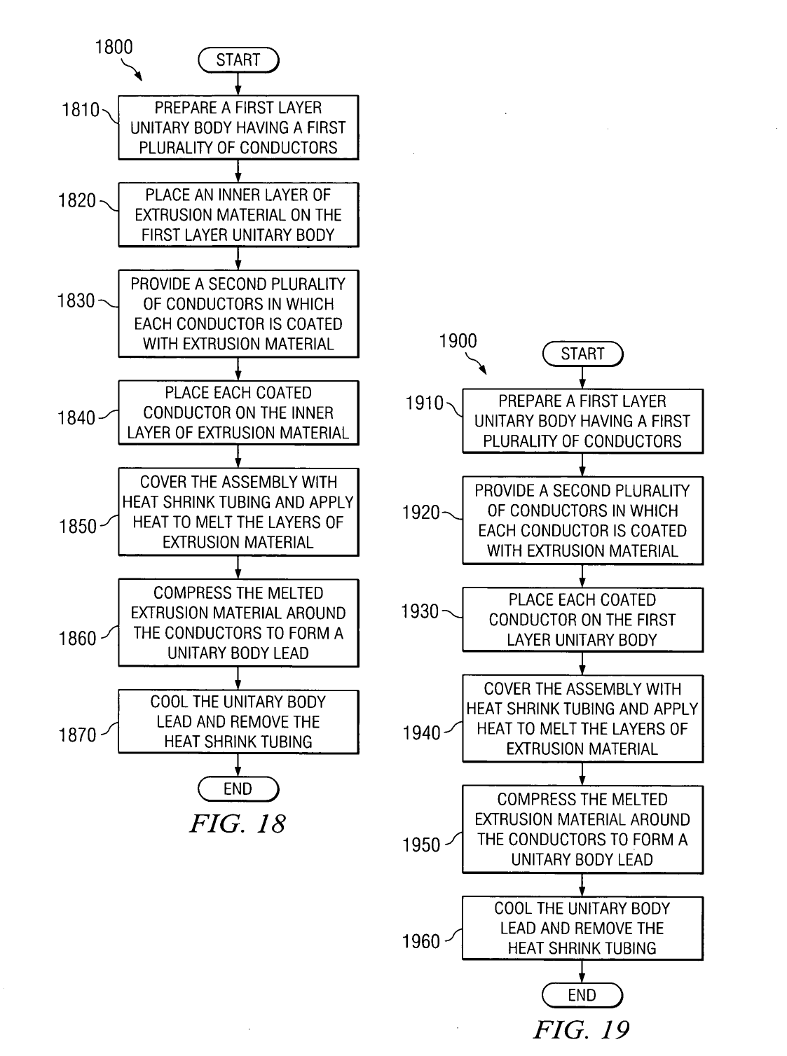

FIG. 18 illustrates a flow chart depicting the steps of an advantageous embodiment of the method of the present invention for making a third embodiment of lead body 120. The steps of the method are collectively referred to with reference numeral 1800.

A first layer unitary body 500 is prepared having a first plurality of conductors 420 (step 1810). An inner layer of extrusion material is placed on the first layer unitary body 500 (step 1820). A second plurality of conductors is provided in which each conductor is coated with extrusion material (step 1830). Each coated conductor is then wrapped around (or placed on) the inner layer of extrusion material (step 1840).

The assembly of the first layer unitary body and the inner layer and the coated conductors is then covered with heat shrink tubing and heat is applied to melt the layers of extrusion material (step 1850). The heat shrink tubing compresses the extrusion material around the conductors to form a unitary body lead (step 1860). The unitary body lead is then cooled and the heat shrink tubing is removed (step 1870).

FIG. 19 illustrates a flow chart depicting the steps of an advantageous embodiment of the method of the present invention for making a fourth embodiment of lead body 120. The steps of the method are collectively referred to with reference numeral 1900.

A first body unitary layer 500 is prepared having a first plurality of conductors 420 (step 1910). A second plurality of conductors is provided in which each conductor is coated with extrusion material (step 1920). Each coated conductor is then wrapped around (or placed on) the first layer unitary body 500 (step 1930). The assembly of the first layer unitary body and the coated conductors is then covered with heat shrink tubing and heat is applied to melt the layers of extrusion material (step 1940). The heat shrink tubing compresses the extrusion material around the conductors to form a unitary body lead (step 1950). The unitary body lead is then cooled and the heat shrink tubing is removed (step 1960).

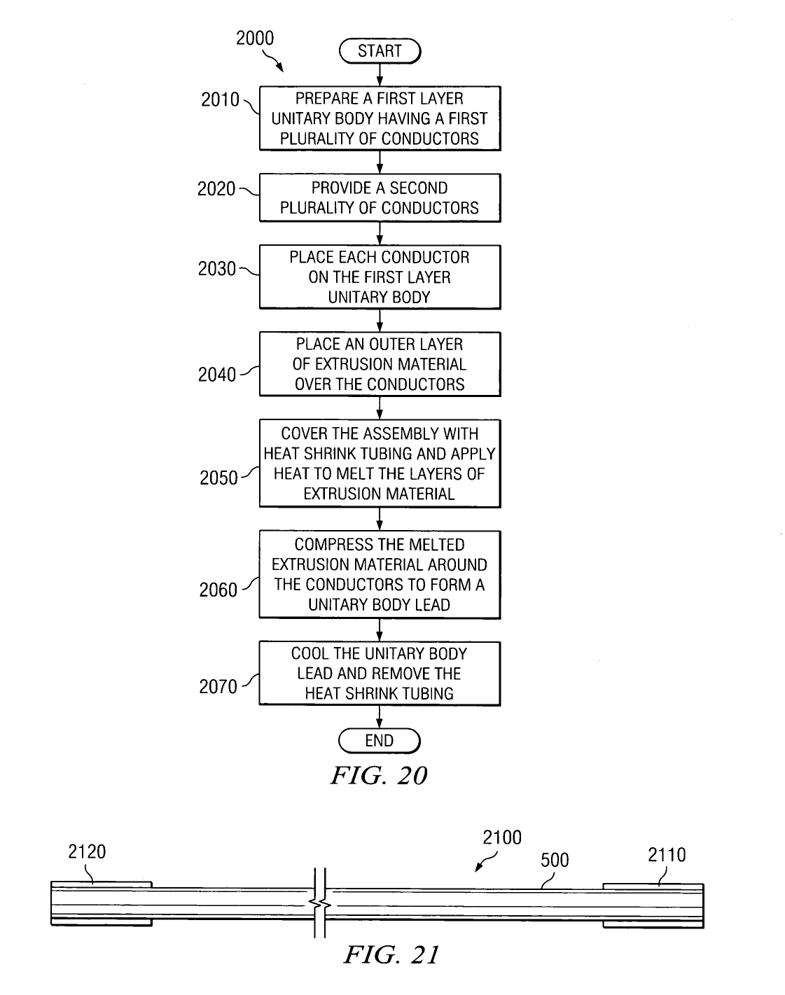

FIG. 20 illustrates a flow chart depicting the steps of one advantageous embodiment of the process of the present invention for making a fifth embodiment of lead body 120. The steps of the method are collectively referred to with reference numeral 2000.

A first body unitary layer 500 is prepared having a first plurality of conductors 420 (step 2010). A second plurality of conductors is provided (step 2020). Each conductor is then wrapped around (or placed on) the first layer unitary body 500 (step 2030). An outer layer of extrusion material is then placed over the second plurality of conductors (step 2040).

The assembly of the first layer unitary body, the conductors and the outer layer is then covered with heat shrink tubing and heat is applied to melt the layers of extrusion material (step 2050). The heat shrink tubing compresses the extrusion material around the conductors to form a unitary body lead (step 2060). The unitary body lead is then cooled and the heat shrink tubing is removed (step 2070).

In order to attach the inner layer of the first plurality of conductors and the second layer of the second plurality of conductors to their respective bands, it is necessary to separate the conductors. In order to separate the conductors, a portion of heat shrink material is placed over the first plurality of conductors at each end of lead body 120 before the second plurality of conductors are placed over first layer unitary body 500. FIG. 21 illustrates a longitudinal cross sectional view of first layer unitary body 500 of the present invention. After heat shrink material has been removed from the entire length of first layer unitary body 500, heat shrink material 2110 is attached to a first end of first layer unitary body 500 and heat shrink material 2120 is attached to a second end of first layer unitary body 500. Then the second plurality of conductors is placed on first layer unitary body 500 and lead body 120 is constructed as previously described.

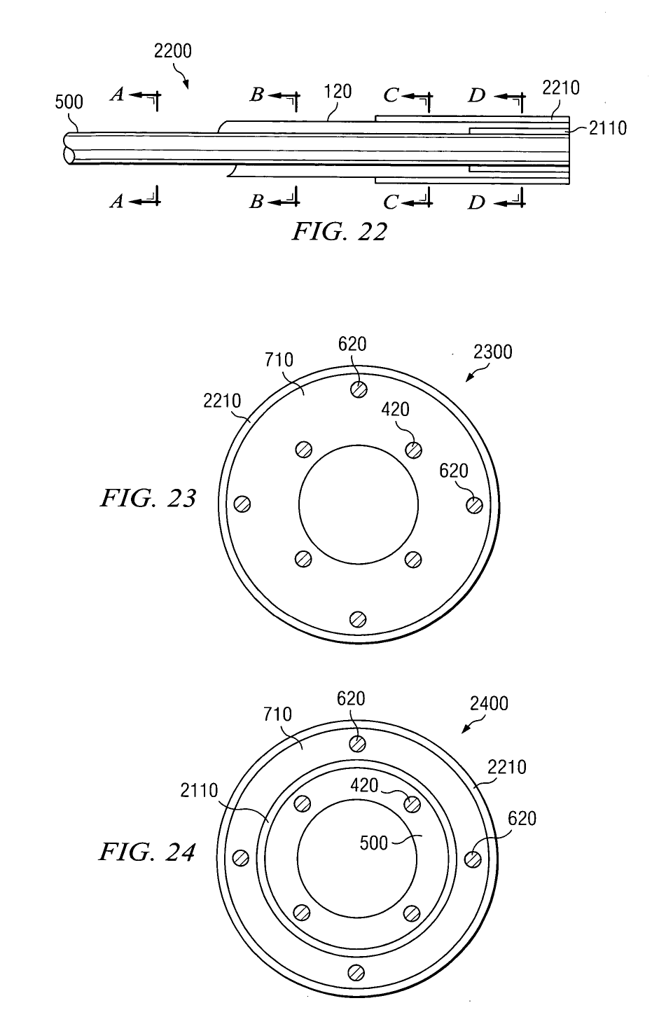

After heat shrink material has been removed from the entire length of lead body 120, heat shrink material 2210 is attached to a first end of lead body 120 and heat shrink material (not shown) is attached to a second end of lead body 120. FIG. 22 illustrates a longitudinal cross sectional view of one end of lead body 120 showing the application of heat shrink material 2110 and heat shrink material 2210 to separate the first and second pluralities of conductors. A cross sectional view along the line A-A of FIG. 22 shows a cross sectional view of first layer unitary body 500. The cross sectional view for line A-A is shown in FIG. 5. A cross sectional view along the line B-B of FIG. 22 shows a cross sectional view of lead body 120. The cross sectional view for line B-B is shown in FIG. 7, and s in FIG. 9, and in FIG. 11, and in FIG. 13 and in FIG. 15.

A cross sectional view along the line C-C of FIG. 22 shows a cross sectional view of lead body 120 covered with heat shrink material 2210. The cross sectional view for line C-C is shown in FIG. 23. FIG. 23 illustrates a cross sectional view 2300 of lead body 120 at a point where lead body 120 is covered with heat shrink material 2210.

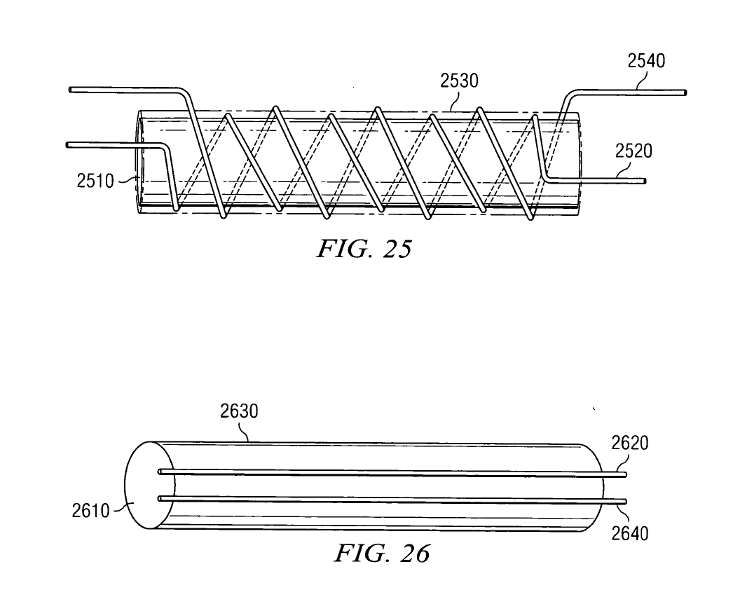

A cross sectional view along the line D-D of FIG. 22 shows a cross sectional view of one end of lead body 120. The cross sectional view for line D-D is shown in FIG. 24. FIG. 24 illustrates a cross sectional view 2400 of lead body 120 at a point where lead body 120 is covered with heat shrink material 2210 and at a point where first layer unitary body 500 is covered with heat shrink material 2110.

The presence of heat shrink material 2110 will not allow the extrusion material between the inner and the outer layers of lead body 120 to bond. After the assembly of lead body 120 is completed, a cutting operation is performed to cut down through the outer layer of lead body 120 to the heat shrink material 2110. The outer layer of lead body 120 is then removed from the end of lead body 120 to expose the first plurality of conductors 420 for electrical attachment to their respective band.

FIG. 25 illustrates a perspective side view of an exemplary mandrel 2510. FIG. 25 illustrates how an exemplary conductor 2520 of a first plurality of conductors may be wound around the axial length of the mandrel 2510 in a first direction within an inner layer of conductors. A cylinder 2530 is shown in dotted outline around mandrel 2510. Cylinder 2530 represents a boundary between an inner layer of conductors (e.g., first layer unitary body 500) and an outer layer of conductors. For clarity, the outer boundary of the outer layer of conductors is not shown in FIG. 25. An exemplary conductor 2540 of a second plurality of conductors may be wound around the axial length of mandrel 2510 within the outer layer of conductors. Exemplary conductor 2540 may be wound in the same direction as conductor 2520 or wound in an opposite direction with respect to the winding of conductor 2520.

Electric current in conductor 2540 may flow in the same direction or in the opposite direction as the electrical current in conductor 2520.

FIG. 26 illustrates a perspective side view of an exemplary mandrel 2610 showing how conductors may be placed lengthwise along the axial length of the mandrel 2610. A first exemplary conductor 2620 is shown placed along the axial length of mandrel 2610 within an inner layer of conductors. A cylinder 2630 is shown in dotted outline around mandrel 2610. Cylinder 2630 represents a boundary between an inner layer of conductors (e.g., first layer unitary body 500) and an outer layer of conductors. For clarity, the outer boundary of the outer layer of conductors is not shown in FIG. 26. A second exemplary conductor 2640 is shown placed along the axial length of mandrel 2610 within the outer layer of conductors. The electrical current in conductor 2640 may flow in the same direction or in the opposite direction as the electrical current in conductor 2620.

It may be advantageous to set forth definitions of certain words and phrases that may be used within this patent document: the terms “include” and “include,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

1. A method for manufacturing a lead body comprising the steps of:
preparing a first layer unitary body comprising a first plurality of conductors;
placing at least one conductor of a second plurality of conductors on said first layer unitary body; and
forming a lead body assembly, wherein the formed lead body assembly comprises a unitary wall and wherein the first plurality of conductors and the at least one conductor of a second plurality of conductors are within the unitary wall.

2. The method as claimed in claim 1 wherein the forming step further comprises using extrusion material in the lead body assembly.

3. The method as claimed in claim 1 wherein the at least one conductor of a second plurality of conductors is coated with a first extrusion material.

4. The method as claimed in claim 1 further comprising the step of placing an inner extrusion layer on the first layer unitary body.

5. The method as claimed in claim 1 further comprising the step of placing an outer extrusion layer on the at least one conductor of a second plurality of conductors.

6. The method as claimed in claim 4 further comprising the step of placing an outer extrusion layer on the at least one conductor of a second plurality of conductors.

7. The method as claimed in claim 6 wherein the inner extrusion layer and the outer extrusion layer are comprised of the same extrusion material.

8. The method as claimed in claim 1 wherein the step of preparing further comprises the step of placing the first layer unitary body comprising a first plurality of conductors on a mandrel.

9. The method as claimed in claim 2 wherein the forming step further comprises the steps of:
placing heat shrink tubing over the lead body assembly;
heating the lead body assembly to melt the extrusion material in the lead body assembly;
compressing the melted extrusion material around the at least one conductor of the second plurality of conductors in the lead body assembly;
cooling the lead body assembly to form the lead body; and
removing the heat shrink tubing from the lead body.

10. A lead for implantation into a human body, the lead comprising:
a unitary lead body assembly comprising:
a unitary wall having an inner portion that forms a lumen;
an inner layer having at least one conductor; and
an outer layer having at least one conductor, wherein the inner layer and the outer layer are within the unitary wall;
at least one electrode located at a distal end of the lead body; and
at least one connector located at a proximal end of the lead body, wherein the at least one connector and the at least one electrode are connected by at least one conductor.

11. The lead as claimed in claim 10 wherein the unitary wall is comprised of extrusion material.

12. The lead as claimed in claim 10, wherein no electrical insulation material is between the conductors and the unitary wall.

13. The lead as claimed in claim 10, wherein the diameter of the lead is no greater than 34 French.

14. The lead as claimed in claim 13, further comprising at least five electrodes.

15. A system for stimulating a portion of a body, wherein the system comprises:
a source for generating a stimulus; and
a lead for receiving the stimulus from the source, wherein the lead comprises:
a unitary lead body assembly comprising:
a unitary wall having an inner portion that forms a lumen;
an inner layer having at least one conductor; and
an outer layer having at least one conductor, wherein the inner layer and the outer layer are within the unitary wall;
at least one electrode located at a distal end of the lead body; and
at least one connector located at a proximal end of the lead body, wherein the at least one connector and the at least one electrode are connected by at least one conductor.

16. The system as claimed in claim 15, wherein the unitary wall comprises extrusion material.

17. The system as claimed in claim 15, wherein no electrical insulation material is between the conductors and the unitary wall.

18. The system as claimed in claim 15, wherein the diameter of the lead is no greater than 34 French.

19. The system as claimed in claim 15, wherein the lead comprises at least five electrodes.

20. The system as claimed in claim 15 wherein the conductors are spirally wound around the lumen.

21. A method for manufacturing a lead body comprising the steps of:
preparing a first layer unitary body comprising a first plurality of conductors;
placing at least one conductor of a second plurality of conductors on said first layer unitary body; and
placing extrusion material over the at least one conductor of the second plurality of conductors to form a lead body assembly.

22. The method as claimed in claim 21 further comprising the steps of:
placing heat shrink tubing over the lead body assembly;
heating the lead body assembly to melt the extrusion material in the lead body assembly;
compressing the melted extrusion material around the at least one conductor of the second plurality of conductors in the lead body assembly;
cooling the lead body assembly to form the lead body; and
removing the heat shrink tubing from the lead body.

23. The method as claimed in claim 21 wherein said at least one conductor of said second plurality of conductor is coated with a layer of extrusion material.

24. A method for manufacturing a lead body comprising the steps of:
preparing a first layer unitary body comprising a first plurality of conductors; and
placing at least one conductor of a second plurality of conductors coated with a layer of extrusion material on the first layer unitary body.

25. A lead body assembly comprising:
a first layer unitary body comprising a first plurality of conductors;
an inner layer of extrusion material on the first unitary body;
a second plurality of conductors wherein each conductor of the second plurality of conductors is coated with a layer of extrusion material and wherein each conductor of the second plurality of conductors is placed on the inner layer of extrusion material; and
an outer layer of extrusion material placed over the second plurality of conductors.

26. A lead body assembly as claimed in claim 25 that has been subjected to heat and compression to form a lead body.

27. A system for stimulating a portion of a body, wherein the system comprises:
a source for generating a stimulus; and
a lead for receiving the stimulus from the source, wherein the lead comprises a lead body formed from a lead body assembly comprising:
a first layer unitary body comprising a first plurality of conductors;
an inner layer of extrusion material on the first unitary body;
a second plurality of conductors wherein each conductor of the second plurality of conductors is coated with a layer of extrusion material and wherein each conductor of the second plurality of conductors is placed on the inner layer of extrusion material; and
an outer layer of extrusion material placed over the second plurality of conductors.

28. A method of manufacturing a lead body comprising the steps of:
placing on a mandrel a first layer unitary body comprising a first plurality of conductors; and
placing at least one conductor of a second plurality of conductors coated with a layer of extrusion material on said first layer unitary body to form a lead body assembly.

29. The method as claimed in claim 28 further comprising the steps of:
placing heat shrink tubing over the lead body assembly;
heating the lead body assembly to melt the extrusion material in the lead body assembly;
compressing the melted extrusion material around the at least one conductor of the second plurality of conductors coated with a layer of extrusion material in the lead body assembly;
cooling the lead body assembly to form the lead body; and
removing the heat shrink tubing from the lead body.

30. The method of manufacturing a lead body as claimed in claim 28 further comprising the steps of:
placing an inner layer of extrusion material on the first layer unitary body; and
placing at least one conductor coated with a layer of extrusion material on the inner layer to form a lead body assembly.

31. The method as claimed in claim 30 further comprising the steps of:
placing heat shrink tubing over the lead body assembly;
heating the lead body assembly to melt the extrusion material in the lead body assembly;
compressing the melted extrusion material around the at least one conductor of the second plurality of conductors coated with a layer of extrusion material in the lead body assembly;
cooling the lead body assembly to form the lead body; and
removing the heat shrink tubing from the lead body.

32. The method of manufacturing a lead body as claimed in claim 30 further comprising the step of:
placing an outer layer of extrusion material on the at least one conductor of the second plurality of conductors coated with a layer of extrusion material to form a lead body assembly.

33. The method as claimed in claim 32 further comprising the steps of:
placing heat shrink tubing over the lead body assembly;
heating the lead body assembly to melt the extrusion material in the lead body assembly;
compressing the melted extrusion material around the at least one conductor of the second plurality of conductors coated with a layer of extrusion material in the lead body assembly;
cooling the lead body assembly to form the lead body; and
removing the heat shrink tubing from the lead body.

34. The method of manufacturing a lead body as claimed in claim 28 further comprising the step of:
placing an outer layer of extrusion material on the at least one conductor of the second plurality of conductors coated with a layer of extrusion material to form a lead body assembly.

35. The method as claimed in claim 34 further comprising the steps of:
placing heat shrink tubing over the lead body assembly;
heating the lead body assembly to melt the extrusion material in the lead body assembly;
compressing the melted extrusion material around the at least one conductor of the second plurality of conductors coated with a layer of extrusion material in the lead body assembly;
cooling the lead body assembly to form the lead body; and
removing the heat shrink tubing from the lead body.

36. A lead body comprising:
a first layer unitary body comprising a first plurality of conductors; and
a second plurality of conductors in which each conductor of the second plurality of conductors is coated with a layer of extrusion material.
